(19) United States
(12) Patent Application Publication
(10) Pub. No.: US 2008/0188830 A1
(43) Pub. Date: Aug. 7, 2008

(54) Selectively reinforced medical devices

(75) Inventors: Joel Rosenblatt, Pottstown, PA (US); Michael E. Starsinic, Williamsport, MD (US); Hiep Do, Sinking Spring, PA (US)

Correspondence Address: AMSTER, ROTHSTEIN & EBENSTEIN LLP, 90 PARK AVENUE, NEW YORK, NY, 10016, US

(73) Assignee: Arrow International, Inc.

(21) Appl. No.: 11/708,162

(22) Filed: Feb. 6, 2007

(51) Int. Cl. A61M 25/00

(52) U.S. Cl. 604/523

(57) ABSTRACT

A medical device including a component made of polyurethane having a reinforced pattern comprising polytetramethylene ether glycol.

### TECHNICAL FIELD

The present invention relates to medical devices, and more particularly to medical devices suitable for at least partial implantation into a body. More specifically, the present invention relates to catheters and other medical devices having portions that are selectively reinforced.

### BACKGROUND

Plasticizing agents are known to make high molecular polymers easier to melt process. This means that plasticizing agents make polymers easier to process at a set temperature or allow polymers to be processed at lower temperatures than would ordinarily be the case without the plasticizing additive. Upon solidification, unless the plasticizing agent is volatile or extracted, the processed polymeric article generally becomes softer, more ductile and weaker.

The use of polytetramethylene ether glycol (PTMEG) as a monomer in polyurethane block copolymers is well known. For example, U.S. Pat. No. 6,992,138 to Tsuji et al. describes the use of PTMEGs as chain extenders in urethane polymerization. Further, U.S. Pat. No. 6,451,005 to Saitou et al. and U.S. Pat. No. 6,616,601 to Hayakawa disclose the use of PTMEGs as the soft segments in ester-ether and polyurethane copolymers, respectively. Other patents specifically disclose the user of polyurethanes incorporating PTMEG as the soft segments of multi-lumen catheters. For example, U.S. Pat. No. 5,226,899 to Lee et al. describes a catheter containing a stripe consisting of relatively hard ester-ether elastomers encapsulated by relatively soft urethane copolymers containing PTMEG segments. In general, softness and flexibility appear to be the recurring theme associated with the use of PTMEGs in conjunction with polyurethanes.

### SUMMARY OF THE INVENTION

In various exemplary embodiments of the present invention, it is recognized that PTMEGs, upon solidification, act to stiffen and mechanically reinforce polyurethane articles, and in particular PTMEGs may be used to increase the flexural modulus of portions of a polyurethane medical device.

A medical device according to an exemplary embodiment of the present invention includes a component made of polyurethane having a reinforced pattern comprising polytetramethylene ether glycol.

A method of forming a medical device according to an exemplary embodiment of the present invention includes forming polyurethane into a component of the medical device, and forming a reinforced pattern in the component, the reinforced pattern comprising polytetramethylene ether glycol.

In at least one embodiment, the medical device is a catheter.

In at least one embodiment, the component is a main body portion of the catheter comprising at least one lumen and the main body portion has a proximal end portion and a distal end portion.

In at least one embodiment, the reinforced pattern comprises at least one strip extending longitudinally from the proximal end portion to the distal end portion of the main body portion of the catheter.

In at least one embodiment, the reinforced pattern is disposed at the distal end portion of the main body portion of the catheter.

In at least one embodiment, the reinforced pattern is disposed at the proximal end portion of the main body portion of the catheter.

In at least one embodiment, the reinforced pattern is a blend of the polytetramethylene ether glycol and the polyurethane, with the polytetramethylene ether glycol being present in the blend at 1% to 5% by weight.

In at least one embodiment, the reinforced pattern is formed by coextrusion of the polytetramethylene ether glycol with the polyurethane.

In at least one embodiment, the reinforced pattern is formed by blending the polytetramethylene ether glycol with the polyurethane.

In at least one embodiment, the reinforced pattern is formed by exposing the component to the polytetramethylene ether glycol after formation of the component.

These and other features of this invention are described in, or are apparent from, the following detailed description of various exemplary embodiments of this invention.

### BRIEF DESCRIPTION OF THE DRAWINGS

Various exemplary embodiments of this invention will be described in detail, with reference to the following figures, wherein:

FIG. 1 shows a catheter according to an exemplary embodiment of the present invention;

FIG. 2 shows a catheter according to another exemplary embodiment of the present invention;

FIG. 3 is a chart of time v. concentration showing the result of an experiment conducted to demonstrate the effectiveness of PTMEG in retarding release of bioactive materials; and

FIG. 4 is a chart of time v. percent release showing the result of an experiment conducted to demonstrate the effectiveness of PTMEG in retarding release of bioactive materials.

### DETAILED DESCRIPTION OF PREFERRED EMBODIMENTS

Various exemplary embodiments of the present invention are directed to a medical device having at least a portion made of polyurethane reinforced by PTMEG. Although the reinforced medical device discussed herein is a catheter, it should be appreciated that the present invention is not limited to catheters, and other medical devices, such as, for example, catheter balloons, stent covers and vascular grafts, are applicable. Further, the present invention is not meant to be limited to any specific type of catheter, and the catheter structures described herein are intended to be merely exemplary.

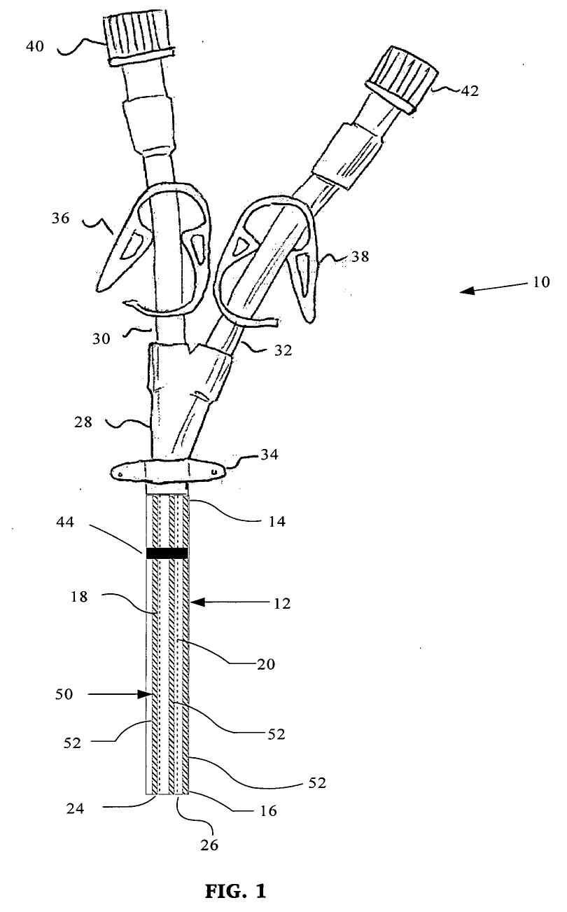

FIG. 1 shows a catheter, generally designated by reference number 10, according to an exemplary embodiment of the present invention. The catheter 10 is a dialysis catheter, including a main body 12 having a proximal end 14 and a distal end 16. First and second lumens 18, 20 extend through the main body 12 and exit through respective ports 24, 26. The proximal end 14 of the catheter main body 12 is secured to a connector hub 28. A first connector tube 30 and a second connector tube 32 extend from the connector hub 28. The connector hub 28 couples the first connector tube 30 to the first lumen 18 for communication therewith, and couples the second connector tube 32 to the second lumen 20 for communication therewith. A suture wing 34 may be rotatably secured to the connector hub 28 to allow the connector hub 28 to be secured to the patient's skin. In addition, a pair of clamps 36 and 38 may be secured over the connector tubes 30 and 32, respectively, for selectively closing off the connector tubes 30, 32 before and after each hemodialysis procedure. A pair of luer lock connector fittings 40 and 42 are secured to the free ends of the connector tubes 30 and 32, respectively, to allow the catheter 10 to be interconnected with fluid infusion lines, aspiration lines, or with the blood inlet and blood return ports of a hemodialysis machine. In the latter case, the first lumen 18 is coupled, via first connector tube 30 and luer lock fitting 40, to an aspiration port of a hemodialysis machine to withdraw blood containing toxins from a blood vessel; and the second lumen 20 is coupled, via second connector tube 32 and luer lock fitting 42, to a cleaned blood return port of the hemodialysis machine to return cleaned blood to the blood vessel. The catheter 10 may also include a stabilizing cuff 44 affixed to an outer portion of the catheter 10 near the proximal end 14.

As shown in FIG. 1, a reinforced pattern, generally designated as reference number 50, is formed in the main body 12 of the catheter 10. In the present embodiment, the reinforced pattern 50 includes reinforced strips 52 that extend longitudinally from the proximal end 14 to the distal end 16 of the main body 12 of the catheter 10. The reinforced strips 52 exhibit greater stiffness than the portions of the main body 12 between the reinforced strips 52, thereby imparting the entire catheter main body 12 with increased overall stiffness. The catheter main body 12 is preferably formed of polyurethane, and the reinforced strips 52 preferably include PTMEG, which imparts the reinforced strips 52 with increased stiffness.

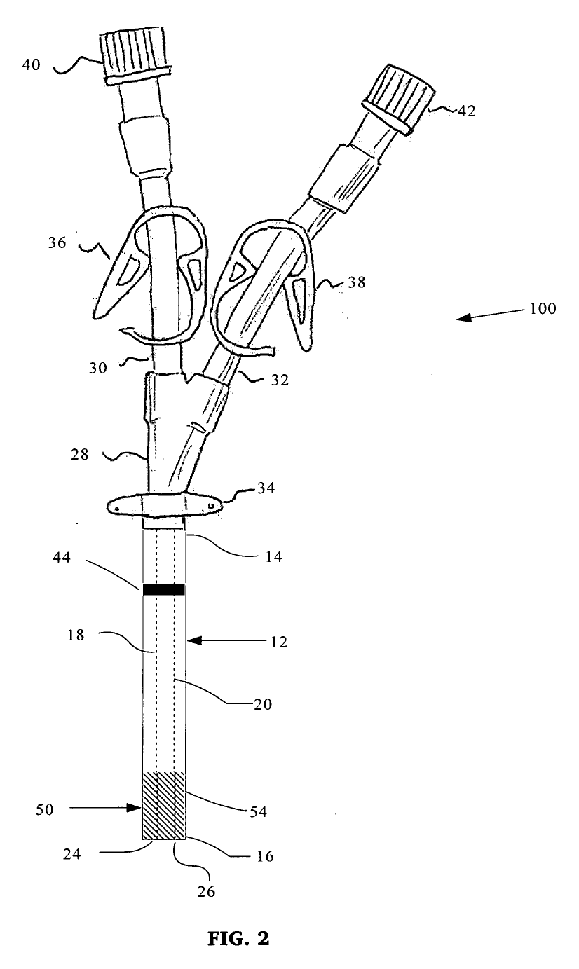

It should be appreciated that the reinforced pattern 50 is not limited to a stripe pattern, and a pattern made up of any number and variety of shapes of reinforced portions may be formed in the catheter main body 12. For example, FIG. 2 shows a catheter 100 according to another exemplary embodiment of the present invention, in which the reinforced pattern 50 is a single portion 54 formed at the distal end 16 of the catheter main body 12. A single reinforced portion formed at the distal end of a catheter would allow the catheter to be more easily inserted into the body of a patient. The reinforced pattern 50 may also be formed at the proximal end 14 of the catheter main body 12.

The reinforced pattern 50 in the catheter body 12 may be formed using any suitable method, such as, for example, melt blending PTMEG with polyurethane prior to extrusion, co-extruding PTMEG with polyurethane, or exposing the already extruded polyurethane to PTMEG.

As discussed above, the reinforced pattern 50 preferably includes PTMEG. According to various exemplary embodiments of the present invention, the incorporation of PTMEG in polyurethanes may serve as a means of introducing valuable functional properties. In particular, the strong compatibility of PTMEG and polyurethanes allows the PTMEG to act as a blending block for block copolymers that can impart different properties to polyurethanes. For example, fluorinated functionality can be introduced using block copolymers of PTMEG and fluoroalkyl side chains. Block copolymers of PTMEG may also be synthesized that contain functionalizable side chains that may be used to bind or covalently tether bioactive molecules. In this regard, the ether group in PTMEG is capable of hydrogen bonding with bioactive molecules and providing slow release.

Example 1 provided below illustrate the increase in stiffness resulting from incorporation of PTMEG into polyurethane extrusions.

### EXAMPLE 1

About half the length of twelve 4 Fr peripherally inserted central catheters (PICCs) were immersed in methanol at room temperature for approximately 18 hours. A solution of 25% by weight Terathane®2000 in methanol was prepared by dissolving the Terathane®2000 under agitation at 50° C. and cooling to room temperature. A shallow layer of the solution was transferred to a metal pan and the exposed portions of the twelve catheters were transferred to the pan containing the solution such that half the length of each catheter was exposed to the Terathane®2000/methanol solution. Three samples were removed after 15, 30, 60 and 120 minutes, respectively. The exposed sections were wiped with a cloth moistened with methanol to remove any solid residue and conditioned 72 hours at ambient temperatures. Flexural moduli of the exposed and unexposed sections of the catheters were tested using ASTM D790 (three-point bend test). The average and standard deviations of the results are presented in Table 1.

TABLE 1

| | Flexural Modulus (psi) | Flexural Modulus (psi) |
|---|---|---|
| Exposure Time (min) | Unexposed Section | Exposed Section |
| 0 | 13412 | — |
| | SD 256 | |
| 15 | 13251 | 15133 |
| | SD 211 | SD 861 |
| 30 | 13096 | 17305 |
| | SD 177 | SD 1030 |
| 60 | 13535 | 17979 |
| | SD 288 | SD 1084 |
| 120 | 13760 | 15918 |
| | SD 223 | SD 3887 |

The data in Table 1 shows that exposure of a portion of the catheter bodies to the Terathane®2000 solution results in increases in the flexural modulus of that section of the catheter over that of the unexposed section of the catheter. In particular, exposure for 30-60 minutes appears to result in the most reliable increase in flexural modulus.

In order to test the permanence of the incorporation of the Terathane®2000 and the stiffening effect, the exposed and unexposed catheter sections were soaked in water at ambient conditions for one week. Flexural modulus of the catheter sections were then re-measured. The results are presented in Table 2.

TABLE 2

| | Flexural Modulus (psi) | Flexural Modulus (psi) |
|---|---|---|
| Exposure Time (min) | Unexposed Section | Exposed Section |
| 0 | 9258 | — |
| | SD 562 | |
| 15 | 9542 | 13882 |
| | SD 491 | SD 1603 |
| 30 | 8966 | 16643 |
| | SD 512 | SD 1808 |
| 60 | 8524 | 16365 |
| | SD 530 | SD 1741 |
| 120 | 9257 | 13071 |
| | SD 503 | SD 1269 |

The data in Table 2 shows that the flexural modulus of all the catheter segments was reduced by exposure to water. However, the flexural moduli of the sections exposed to the Terathane®2000 remained significantly higher than that of the unexposed sections. Further, the minimum observed reduction in the flexural modulus of the unexposed catheter is about 28%, while the maximum observed reduction in the flexural modulus of the exposed section is about 18%. This shows that the incorporation of Terathane®2000 and the resulting stiffening effect on the catheter body was durable.

As discussed above, PTMEG is capable of providing extended release of bioactive molecules, as well as providing a stiffening effect to polyurethane. An example of a bioactive ingredient that may be slow released from PTMEG is an antimicrobial, such as, for example, chlorhexidine (CHX). Different antimicrobial agents can be used with the present invention. Examples include, but are not limited to, a guanidium (e.g., chlorhexidine, alexidine, and hexamidine), a biguanide, a bipyridine (e.g., octenidine), a phenoxide antiseptic (e.g., colofoctol, chloroxylenol, and triclosan), an alkyl oxide, an aryl oxide, a thiol, an aliphatic amine, an aromatic amine and halides such as F⁻, Br⁻ and I⁻, and salts thereof. Additional examples include bismuth (and antimicrobial bismuth compounds), chlorxylenol, protamine, gendine, genlenol, genlosan, genfoctol, silver (and antimicrobial silver compounds, such as silver sulfadiazine and chlorhexidine-silver sulfadiazine), chlorhexidine acetate, chlorhexidine gluconate, chlorhexidine hydrochloride, chlorhexidine and propanol, chlorhexidine base and chlorhexidine acetate, povidone-iodine, cefazolin, teicoplanin, vancomycin, an antimicrobial dye, and antimicrobial mixtures containing carbon and platinum. The antimicrobial dye can be, for example, a triarylmethane dye, a monoazo dye, a diazo dye, an indigoid dye, a xanthene dye, a fluorescein dye, an anthraquinone dye or a quinoline dye. More specific examples of dyes include gentian violet, crystal violet, ethyl violet, brilliant green, and methylene blue. Furthermore, different antibiotics or mixtures of antibiotics can be used with the present invention. A preferred mixture of antibiotics inhibits bacterial growth by different mechanisms, e.g., a DNA or RNA replication inhibitor combined with a protein synthesis inhibitor. Examples of agents that inhibit bacteria by inhibiting DNA or RNA replication include rifampicin, taurolidone, 5-fluorouracil, and Adriamycin. Examples of agents that inhibit protein synthesis include tetracyclines, e.g. minocycline, and clindamycin. Another category of an antimicrobial agent is quorum sensing inhibitors such as inhibitors of derivatives of Autoinducer 1 (N-acyl homoserine lactone) and Autoinducer 2 (furanosyl borate diester), inhibitors of their receptors, and inhibitors of the genes and kinases involved in their upregulation. Examples of quorum sensing inhibitors include furanones, including halogenated furanones. Still another category of an antimicrobial agent is a host-defense protein or peptide, such as an aminosterol or a magainin, or a mimetic thereof. Additional examples of antimicrobial agents can be found, e.g., in U.S. Pat. Nos. 5,221,732, 5,643,876, 5,840,740, 6,303,568, 6,388,108, and 6,875,744, in U.S. Patent Application Publication No. 2003/0078242, and in PCT International Publication No. WO 2004/099175, the contents of which are incorporated by reference. Preferably, the antimicrobial agent contains chlorhexidine (including the free base and salts thereof and mixtures of the free base and salts).

The use of solvents to dissolve CHX and PTMEG does not retard release of CHX when solvent evaporation is conducted at ambient temperatures. However, when the solution is conditioned at elevated temperatures, release kinetics are found to be significantly retarded. Example 2 provided below illustrates the effectiveness of PTMEG in providing extended release of CHX.

### EXAMPLE 2

### A. CHX Loaded Terathane Via Suspension

Sample 1: Suspension of CHX in Terathane (20% loading)

1.0085 g of CHX was manually mixed with 5.0376 g molten Terathane (MW: 2000). 0.10919 g of CHX-Terathane molten mixture was added in a 50 ml conical flask. The coating technique was done by rolling the flask to allow the Terathane to uniformly coat the wall surface of the flask until the molten mixture solidified.

### B. CHX Loaded Terathane Via Methyl Ethyl Ketone Solvent

5% w/v CHX in methyl ethyl ketone (MEK) was prepared by dissolving 1.0067 g of CHX in 20 ml of methyl ethyl ketone at room temperature. This CHX solution was then mixed with 20 g of molten Terathane.

### Sample 2: Evaporate MEK Solvent at Room Temperature

0.53238 g of CHX-MEK-Terathane mixture was coated on the inside of a 50 ml conical flask at room temperature by rolling the flask on a flat flask surface until the molten mixture solidified or most MEK evaporated. Most of the coating formed on the bottom of the flask.

### Sample 3. Evaporate MEK Solvent at 65° C.

0.50882 g of CHX-MEK-Terathane mixture was added in a 50 ml conical flask and kept in a 65° C. water bath until most MEK evaporated. The flask containing CHX loaded molten was rolled on a flat surface until the Terathane solidified. Most of the coating formed on the bottom of the flask.

Table 3 below summarizes the conditions of the three samples.

TABLE 3

| | | Theoretical CHX | Process |
|---|---|---|---|
| Sample ID | Sample Size (g) | from sample (mg) | condition |
| 1 | 0.10919 | 21.8 | Molten suspension |
| 2 | 0.53238 | 21.3 | R.T. solution |
| 3 | 0.50882 | 20.4 | 65° C. solution |
| 4 | | 20.0 | CHX only |

The release study consisted of the three coated tubes along with a 4^{th} control tube containing 20 mg of chlorhexidine base. 45 ml of deionized water was added to each tube. After one hour the water from each tube was decanted into another 50 mL polypropylene tube and capped until analysis. A fresh 45 ml of DI water was added to each tube and the process was repeated at 3 and 5 hours, 1, 2, 3, 4 and 7 days. The control tube was centrifuged at the end of each time period and the water was removed via pipette, rather than decanting, to ensure no CHX particles were suspended or transferred to the sample tube. Analysis of the samples was done following the completion of the release study on a UV-Vis spectrophotometer at a wavelength of 253 nm. Samples were diluted as necessary to obtain values within the standard curve.

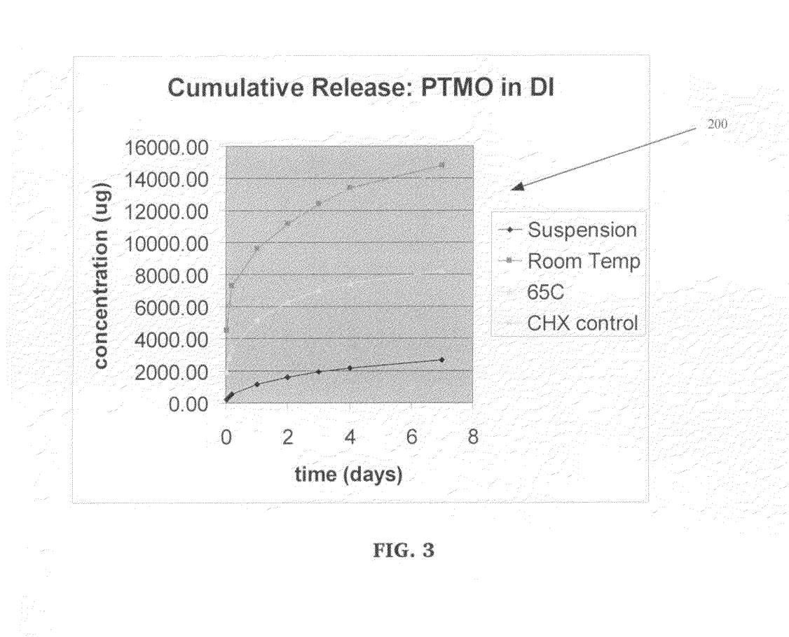

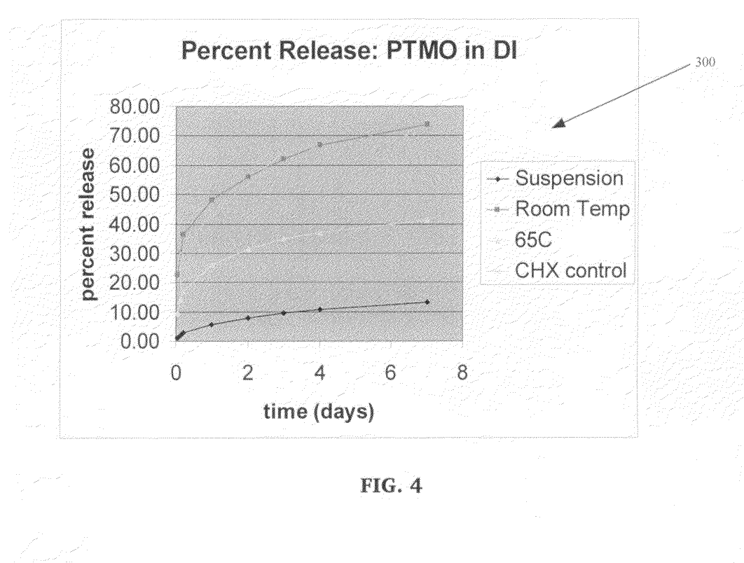

The results of the above experiment are present in FIGS. 3 and 4. FIG. 3 shows a chart 200 of time v. concentration, and FIG. 4 shows a chart 300 of time v. percent release. Charts 200 and 300 show that Sample 1 (CHX loaded Terathane via suspension) releases the lowest concentration and percentage of CHX over time. Further, charts 200 and 300 show that there is no significant retarded release of CHX from Sample 2 (solvent evaporated at room temperature), but there is significant retarded release from Sample 3 (solvent evaporated at elevated temperature).

In various exemplary embodiments of the present invention, antithrombogenic and/or anti-inflammatory agents may be added to a medical device having at least a portion made of polyurethane reinforced by PTMEG. Such agents may include platelet inhibitors, thrombin inhibitors and fibrinolytics, for example. Anti-inflammatory agents include agents that suppress fibrous sheath formation around the medical device, such as antifibrotics, M-TOR inhibitors, steroids and antineoplastic agents. Also, the medical device may be surface coated with antimicrobial and/or antithrombogenic agents or polymers, as disclosed in U.S. patent application Ser. No. 11/293,056, entitled Catheter With Polymeric Coating, incorporated herein by reference.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A medical device comprising:
a component made of polyurethane having a reinforced pattern comprising polytetramethylene ether glycol.

2. The medical device of claim 1, wherein the medical device is a catheter.

3. The medical device of claim 2, wherein the component is a main body portion of the catheter comprising at least one lumen and the main body portion has a proximal end portion and a distal end portion.

4. The medical device of claim 3, wherein the reinforced pattern comprises at least one strip extending longitudinally from the proximal end portion to the distal end portion of the main body portion of the catheter.

5. The medical device of claim 3, wherein the reinforced pattern is disposed at the distal end portion of the main body portion of the catheter.

6. The medical device of claim 3, wherein the reinforced pattern is disposed at the proximal end portion of the main body portion of the catheter.

7. The medical device of claim 1, wherein the reinforced pattern is a blend of the polytetramethylene ether glycol and the polyurethane, with the polytetramethylene ether glycol being present in the blend at 1% to 5% by weight.

8. The medical device of claim 1, further comprising an extended release material incorporated in the component.

9. The medical device of claim 8, wherein the release material is mixed with the polytetramethylene ether glycol.

10. The medical device of claim 9, wherein the extended release material is dissolved in the polytetramethylene ether glycol using a solvent at a temperature above ambient temperature.

11. The medical device of claim 8, wherein the extended release material is an antimicrobial agent.

12. The medical device of claim 11, wherein the antimicrobial agent comprises any one or more of a guanidium, a biguanide, a bipyridine, a phenoxide antiseptic, an alkyl oxide, an aryl oxide, a thiol, an aliphatic amine, an aromatic amine, bismuth, chlorxylenol, protamine, colofoctol, chloroxylenol, triclosan, gendine, genlenol, genlosan, genfoctol, octenidine, chlorhexidine, alexidine, hexamidine, silver, silver sulfadiazine, chlorhexidine-silver sulfadiazine, chlorhexidine acetate, chlorhexidine gluconate, chlorhexidine hydrochloride, chlorhexidine and propanol, chlorhexidine base and chlorhexidine acetate, povidone-iodine, cefazolin, teicoplanin, vancomycin, an aminosterol, a magainin, a furanone, a halogenated furanone, a triarylmethane dye, a monoazo dye, a diazo dye, an indigoid dye, a xanthene dye, a fluorescein dye, an anthraquinone dye, a quinoline dye, gentian violet, crystal violet, ethyl violet, brilliant green, methylene blue, rifampicin, taurolidone, 5-fluorouracil, Adriamycin, a tetracycline, minocycline, clindamycin, rifampin-minocycline, and salts thereof.

13. The medical device of claim 12, wherein the antimicrobial agent comprises chlorhexidine.

14. The medical device of claim 1, wherein the component comprises at least one of antithrombogenic and anti-inflammatory agents.

15. A method of forming a medical device, comprising:
forming polyurethane into a component of the medical device; and
forming a reinforced pattern in the component, the reinforced pattern comprising polytetramethylene ether glycol.

16. The method of claim 15, wherein the reinforced pattern is formed by coextrusion of the polytetramethylene ether glycol with the polyurethane.

17. The method of claim 16, wherein the reinforced pattern is formed by blending the polytetramethylene ether glycol with the polyurethane.

18. The method of claim 15, wherein the reinforced pattern is formed by exposing the component to the polytetramethylene ether glycol after formation of the component.

19. The method of claim 15, wherein the medical device is a catheter.

20. The method of claim 19, wherein the component is a main body portion of the catheter comprising at least one lumen and the main body portion has a proximal end portion and a distal end portion.

21. The method of claim 20, wherein the step of forming a reinforced pattern comprises forming at least one reinforced strip extending longitudinally from the proximal end portion to the distal end portion of the main body portion of the catheter.

22. The method of claim 20, wherein the step of forming a reinforced pattern comprises forming the reinforced pattern at the distal end portion of the main body portion of the catheter.

23. The method of claim 20, wherein the step of forming a reinforced pattern comprises forming the reinforced pattern at the proximal end portion of the main body portion of the catheter.

24. The method of claim 15, further comprising incorporating an extended release material into the medical device.

25. The method of claim 24, wherein the step of incorporating comprises mixing the release material with the polytetramethylene ether glycol.

26. The method of claim 24, wherein the step of incorporating comprises dissolving the release material in the polytetramethylene ether glycol using a solvent at a temperature above ambient temperature.

27. The method of claim 24, wherein the extended release material is an antimicrobial agent.

28. The method of claim 27, wherein the antimicrobial agent comprises any one or more of a guanidium, a biguanide, a bipyridine, a phenoxide antiseptic, an alkyl oxide, an aryl oxide, a thiol, an aliphatic amine, an aromatic amine, bismuth, chlorxylenol, protamine, colofoctol, chloroxylenol, triclosan, gendine, genlenol, genlosan, genfoctol, octenidine, chlorhexidine, alexidine, hexamidine, silver, silver sulfadiazine, chlorhexidine-silver sulfadiazine, chlorhexidine acetate, chlorhexidine gluconate, chlorhexidine hydrochloride, chlorhexidine and propanol, chlorhexidine base and chlorhexidine acetate, povidone-iodine, cefazolin, teicoplanin, vancomycin, an aminosterol, a magainin, a furanone, a halogenated furanone, a triarylmethane dye, a monoazo dye, a diazo dye, an indigoid dye, a xanthene dye, a fluorescein dye, an anthraquinone dye, a quinoline dye, gentian violet, crystal violet, ethyl violet, brilliant green, methylene blue, rifampicin, taurolidone, 5-fluorouracil, Adriamycin, a tetracycline, minocycline, clindamycin, rifampin-minocycline, and salts thereof.

29. The method of claim 28, wherein the antimicrobial agent comprises chlorhexidine.

30. The method of claim 15, further comprising adding at least one of antithrombogenic and anti-inflammatory agents to the component.
